(19) United States
(12) Patent Application Publication
(10) Pub. No.: US 2024/0191312 A1
(43) Pub. Date: Jun. 13, 2024

(54) COMPOSITIONS AND KITS FOR DETECTING THE PRESENCE OF A HYPERVIRULENT CLOSTRIDIUM DIFFICILE STRAIN

(71) Applicant: Mobidiag Oy, Espoo (FI)

(72) Inventors: Juha KIRVESKARI, Espoo (FI); Jaakko KURKELA, Espoo (FI)

(73) Assignee: Mobidiag Oy, Espoo (FI)

(21) Appl. No.: 18/587,858

(22) Filed: Feb. 26, 2024

Related U.S. Application Data
(62) Division of application No. 18/087,603, filed on Dec. 22, 2022.
Application No. 18/087,603 is a division of application No. 15/532,132, filed on Jun. 1, 2017, now Pat. No. 11,566,294.

(30) Foreign Application Priority Data
Dec. 19, 2014 (FI) 20146124

(51) Int. Cl. C12Q 1/689

(52) U.S. Cl. CPC C12Q 1/689; C12Q 2600/158; C12Q 2600/16

(57) ABSTRACT

The present invention provides a nucleic acid amplification based method for detecting a hypervirulent Clostridium difficile strain in a biological sample. The present invention is based on the use of oligonucleotide primers and probes specific to negative and positive markers in hypervirulent Clostridium difficile genome.

### CROSS REFERENCE TO RELATED APPLICATIONS

This application is a divisional of U.S. application Ser. No. 18/087,603, filed Dec. 22, 2022, which is a divisional of U.S. patent application Ser. No. 15/532,132, filed Jun. 1, 2017, now issued as U.S. Pat. No. 11,566,294, which is a national phase entry pursuant to 35 U.S.C. § 371 of International Application No. PCT/FI2015/050911, filed Dec. 18, 2015, which claims the benefit of priority of Finnish Provisional Application No. 20146124, filed Dec. 19, 2014. Each of the foregoing applications is incorporated by reference herein in its entirety.

### REFERENCE TO SEQUENCE LISTING

The present application contains a Sequence Listing which has been submitted electronically in ST26 format. Said ST26 Copy, created on Feb. 2, 2024, is named “4342-P6US-DIV2_Seq_Listing_ST26” and is 15,118 bytes in size. The information in the electronic format of the Sequence Listing is incorporated herein by reference it its entirety.

### FIELD

The present invention relates to the field of nucleic acid amplification based diagnostic assays. More specifically, the present invention provides a PCR based method for detecting a hypervirulent Clostridium difficile strain, preferably toxin producing Clostridium difficile strain 027, in a biological sample, such as a stool sample. The present invention is based on the use of oligonucleotide primers and probes specific to negative and positive markers for hypervirulent Clostridium difficile strains.

### BACKGROUND OF THE INVENTION

C. difficile infection (CDI) is a toxin-mediated intestinal disease. The clinical outcomes of CDI can range from asymptomatic colonization to more severe disease syndromes, including severe diarrhoea, abdominal pain, fever and leukocytosis. C. difficile is recognized as the main cause of infectious diarrhoea that develops in patients after hospitalization and antibiotic treatment. Therefore, CDI is now considered to be one of the most important of health care-associated infections. Further, non-hospital-associated reservoirs of C. difficile are also emerging, and C. difficile is capable of spreading in animal hosts (Denéve et al., 2009; Rupnik et al, 2009).

C. difficile testing methods currently include cytotoxigenic culture methods, cytotoxin assays (CYT) detecting the toxins A and B produced by C. difficile, PCR based assays for detection of the tedB gene of C. difficile, and assays for detection of C. difficile-specific glutamate dehydrogenase (GDH) (Eastwood et al., 2009).

In the prior art, the PCR based test have been found to be reliable, sensitive, and specific diagnostic tools for rapid screening and identification of samples containing C. difficile (Eastwood et al., 2009; Hirvonen et al., 2013; Houser et al., 2010 and WO2012087135). In commercial use is a method disclosed by WO2010116290 (Philips) relating to a multiplex PCR assay for the detection of a toxigenic C. difficile strain by analysing the presence or absence of the cytotoxin tcdB gene and deletions in the tedC gene.

Although a number of PCR based assays for detecting toxin producing Clostridium difficile strain are already disclosed, there is still a need in the field for a PCR assay which is able to provide high specificity and reliability for the detection of those C. difficile strains which are hypervirulent. The present inventors have now located DNA sequence regions in Clostridium difficile genome that are surprisingly well-suited for specific and sensitive amplification of negative and positive markers relating to hypervirulent Clostridium difficile strains.

The sample matrix, which in diarrhoea diagnostics is commonly a stool or food sample, is likely to contain a host of PCR inhibitors. This reduces amplification efficiency of the PCR reaction and thus even more careful optimization is expected from the amplicon design step to verify that all templates and copy numbers are amplified equally but also efficiently enough. Hence, oligonucleotide design enabling high PCR efficiency (optimally as close to 100% as possible) is required. The detection method used may also affect amplification efficiency and/or bias.

The present inventors have now located DNA sequence regions that are well suited for specific and sensitive amplification and quantification of diarrhoea causing hypervirulent Clostridium difficile strains. The amplicons have been designed to be so specific that they can be combined into any multiplex sets with each other. Naturally a prerequisite to this is that all the disclosed amplicons have also been designed to amplify in the same reaction and cycling conditions. The aim of the invention is to replace antigen testing and culturing as a screening test for hypervirulent Clostridium difficile, and thus provide process improvements for the laboratory and clinical benefits in improved patient management by providing rapidly a rich set of information. Further, infection control could benefit if clinical microbiology laboratories could readily differentiate between non-toxigenic C. difficile and hypervirulent C. difficile.

### SUMMARY OF THE INVENTION

One object of the present invention is to provide a method of detecting the presence of a hypervirulent Clostridium difficile strain in a biological sample, the method comprising: performing a nucleic acid amplification reaction comprising DNA extracted from the biological sample as a template, a first oligonucleotide primer set specific for amplifying a target sequence in the C. difficile hydR gene in the reaction, wherein said hydR gene comprises a sequence corresponding to SEQ ID NO:1, and a second oligonucleotide primer set specific for amplifying at least part of the target sequence corresponding to C. difficile sequence set forth in SEQ ID NO:2 in the reaction.

Another object of the present invention is to provide an oligonucleotide primer set comprising an oligonucleotide comprising or consisting of at least 10 contiguous nucleotides present in a nucleotide sequence as set forth in SEQ ID NO:3 and an oligonucleotide comprising or consisting of at least 10 contiguous nucleotides present in a nucleotide sequence as set forth in SEQ ID NO: 4, wherein the oligonucleotide primer set amplifies a target sequence in the C. difficile hydR gene.

Another object of the present invention is to provide an oligonucleotide primer set comprising an oligonucleotide comprising or consisting of at least 10 contiguous nucleotides present in a nucleotide sequence as set forth in SEQ ID NO: 5 and an oligonucleotide comprising or consisting of at least 10 contiguous nucleotides present in a nucleotide sequence as set forth in SEQ ID NO: 6, wherein the oligonucleotide primer set amplifies a specific target sequence in C. difficile genome.

Another object of the present invention is to provide an oligonucleotide primer set comprising an oligonucleotide comprising or consisting of at least 10 contiguous nucleotides present in a nucleotide sequence as set forth in SEQ ID NO: 11 and an oligonucleotide comprising or consisting of at least 10 contiguous nucleotides present in a nucleotide sequence as set forth in SEQ ID NO: 12, wherein the oligonucleotide primer set amplifies a target sequence in the C. difficile tcdB gene.

Another object of the present invention is to provide a kit for detecting a hypervirulent Clostridium difficile strain in a biological sample, the kit comprising: an oligonucleotide primer set as defined above; and a reagent for performing amplification of a nucleic acid in a nucleic acid amplification reaction.

### DETAILED DESCRIPTION OF THE INVENTION

The purpose of the method of the present invention is to serve as a primary microbiological screening test for the qualitative identification of hypervirulent C. difficile, and a recurrent disease associated ribotype 027. The method is preferably performed from DNA extracted directly from a biological sample, such as a stool sample, without the use of an enrichment culture. Preferably, the method of the invention is a PCR-based C. difficile assay: such as a qPCR assay, or a qualitative multiplexed nucleic acid-based in vitro diagnostic test intended for detecting of nucleic acid markers corresponding to the detection and identification of hypervirulent Clostridium difficile and toxin producing 027 ribotype selective markers.

As used herein, a “target sequence” present in a nucleic acid sample is a strand of C. difficile DNA to be primed and extended by a “primer”. A target sequence may be either single-stranded or in a duplex with its complementary sequence. Target sequence as defined in the present invention is preferably purified to some degree prior to the amplification reactions described herein.

As used herein, the term “oligonucleotide” refers to any polymer of two or more of nucleotides, nucleosides, nucleobases or related compounds used as a reagent in the DNA amplification methods, such as primers and probes. The oligonucleotide may be DNA and/or RNA and/or analogs thereof. The term oligonucleotide does not denote any particular function to the reagent; rather, it is used generically to cover all such reagents described herein. Specific oligonucleotides of the present invention are described in more detail below. As used herein, an oligonucleotide can be virtually any length, limited only by its specific function in the DNA amplification reaction. Oligonucleotides of a defined sequence and chemical structure may be produced by techniques known to those of ordinary skill in the art, such as by chemical or biochemical synthesis, and by in vitro or in vivo expression from recombinant nucleic acid molecules, e.g., bacterial or viral vectors. Oligonucleotides may be modified in any way, as long as a given modification is compatible with the desired function of a given oligonucleotide. One of ordinary skill in the art can easily determine whether a given modification is suitable or desired for any given oligonucleotide of the present invention. Modifications include, but are not limited to base modifications, sugar modifications or backbone modifications. While design and sequence of oligonucleotides for the present invention depend on their function as described below, several variables must generally be taken into account. Among the most critical are: length, G/C content, melting temperature (Tm), Gibb free energy (G), specificity, self-complementarity and complementarity with other oligonucleotides in the system, polypyrimidine (T, C) or polypurine (A, G) stretches, and the 3′-end sequence. Controlling for these and other variables is a standard and well-known aspect of oligonucleotide design, and various computer programs are readily available to screen large numbers of potential oligonucleotides for optimal ones.

As used herein, the term “PCR reaction”, “PCR amplifying” or “PCR amplification” refers generally to cycling polymerase-mediated exponential amplification of nucleic acids employing primers that hybridize to complementary strands, as described for example in Innis et al, PCR Protocols: A Guide to Methods and Applications, Academic Press (1990). Devices have been developed that can perform thermal cycling reactions with compositions containing fluorescent indicators which are able to emit a light beam of a specified wavelength, read the intensity of the fluorescent dye, and display the intensity of fluorescence after each cycle. The amplification product contains a sequence having sequence identity with a target nucleic acid sequence or its complement and can be detected with, for example, an intercalating dye or a detection probe having specificity for a region of the target nucleic acid sequence or its complement. The PCR reaction as defined in the present invention is preferably performed as a real-time PCR assay.

As used herein, the term “probe” refers to any of a variety of signalling molecules indicative of amplification. For example, SYBR® Green and other DNA-binding dyes are detector probes. Some detector probes can be sequence-based, for example 5′ nuclease probes. Various detector probes are known in the art, for example TaqMan® probes (See U.S. Pat. No. 5,538,848). The melting temperature, Tm, of the probes can be increased by addition of modified nucleotides. The amount of modified nucleotides in one probe is preferably 1, 2, 3, 4 or more. The modified nucleotide can be a LNA nucleotide (Exiqon A/S), minor groove binder (MGB™), SuperBase, or Peptide Nucleic Acid (PNA) or any other modification increasing the Tm of the probe.

A person skilled in the art knows that amplified target sequences, i.e. amplicons, naturally vary in related strains. This minor variation can be taken into account while designing primers suitable to amplify said amplicons in the method of the present invention. Preferably, at least 50, 60, 70, 80, 90 or 100 nucleotides long sequence of each of the target amplicons selected from the group consisting of SEQ ID NOS: 1, 2 and 10 is amplified in the method.

Preferably, the primers and probes comprise the sequences as defined in the claims and are less than 30, 35, 40, 45, 50 or 55 nucleotides long, and more preferably, less than 50 nucleotides long. Each of the present primers and probes can also be defined as consisting of at least 10, 15, 16, 17, 18, 19 or 20 contiguous nucleotides present in any one of primer or probe sequences selected from the group consisting of SEQ ID NOS:3-9 and 11-13 or comprising a sequence selected from the group consisting of SEQ ID NOS:3-9 and 11-13.

The present invention is directed to a method of detecting the presence of a hypervirulent Clostridium difficile strain in a biological sample. Preferably, the method is a real-time PCR assay. The method can be performed using a DNA chip, gel electrophoresis, a radiation measurement, a fluorescence measurement, or a phosphorescence measurement. A person skilled in the art may use the primers and probes of the invention also in other methods and platforms utilizing PCR or nucleic acid amplification. Said biological sample can be, e.g., a stool sample, an environmental sample or a food sample.

The method comprises the step of:
performing a nucleic acid amplification reaction comprising DNA extracted from the biological sample as a template, a first oligonucleotide primer set specific for amplifying a target sequence in the C. difficile hydR gene in the reaction, wherein said hydR gene comprises a sequence corresponding to SEQ ID NO:1, and a second oligonucleotide primer set specific for amplifying at least part of the target sequence corresponding to C. difficile sequence set forth in SEQ ID NO:2 in the reaction. Preferably, the method comprises a step of detecting the presence of a hypervirulent Clostridium difficile strain in said biological sample by any method capable of detecting amplified target sequences in the reaction.

The hypervirulent Clostridium difficile strain is detected in the sample, when the first oligonucleotide primer set does not amplify a specific product, i.e. the target sequence in hydR gene is a negative marker for hypervirulent Clostridium difficile strain, and the second oligonucleotide primer set amplifies a specific product, i.e. the sequence targeted by the second primer set in C. difficile genome is a positive marker for hypervirulent Clostridium difficile strains.

The most important hypervirulent Clostridium difficile strain detected by the present method is toxin producing Clostridium difficile strain 027. Thus, the present method is particularly directed to the detection of this Clostridium difficile strain. The presence of C. difficile hydR gene DNA in said sample, however, indicates that Clostridium difficile strain 027 is not present in the examined sample or that in addition to the presence of a toxin producing Clostridium difficile strain 027 there is also presence of another Clostridium difficile strain in the sample. A skilled person of the art is, however, aware that some of hypervirulent C. difficile strains are not classified as 027-ribotype strains, therefore, the present invention is also directed to the detection of hypervirulent 027-ribotype-resembling Clostridium difficile strains.

Preferably, the first oligonucleotide primer set targets the C. difficile hydR gene and amplifies the hydR sequence set forth in SEQ ID NO:1 so that at least part of the sequence is specifically amplified in the amplification reaction. More preferably, the first oligonucleotide primer set comprises an oligonucleotide comprising or consisting of at least 10 contiguous nucleotides present in the nucleotide sequence as set forth in SEQ ID NO: 3 and an oligonucleotide comprising or consisting of at least 10 contiguous nucleotides present in the nucleotide sequence as set forth in SEQ ID NO: 4, said primers amplifying at least part of the hydR sequence set forth in SEQ ID NO:1. Most preferably, the first oligonucleotide primer set comprises an oligonucleotide comprising or consisting of the nucleotide sequence as set forth in SEQ ID NO: 3 and an oligonucleotide comprising or consisting of the nucleotide sequence as set forth in SEQ ID NO: 4.

The presence of the target sequence amplified with the first oligonucleotide primer set can be detected by the use of a probe comprising or consisting of at least 10 contiguous nucleotides present in the nucleotide sequence as set forth in SEQ ID NO:7, or preferably, by the use of a probe comprising or consisting of the nucleotide sequence as set forth in SEQ ID NO:7.

The target sequence of the second oligonucleotide primer set in C. difficile genome corresponds to a gene encoding a putative conjugative transposon DNA recombination protein. Preferably, said second oligonucleotide primer set comprises an oligonucleotide comprising or consisting of at least 10 contiguous nucleotides present in a nucleotide sequence as set forth in SEQ ID NO: 5 and an oligonucleotide comprising or consisting of at least 10 contiguous nucleotides present in a nucleotide sequence as set forth in SEQ ID NO: 6. More preferably, the second oligonucleotide primer set comprises an oligonucleotide comprising or consisting of the nucleotide sequence as set forth in SEQ ID NO: 5 and an oligonucleotide comprising or consisting of the nucleotide sequence as set forth in SEQ ID NO: 6.

The probes for the second oligonucleotide primer set as defined in SEQ ID NO: 8 and 9 can be used as competitive probes in a same reaction to detect a G/A polymorphism in C. difficile genome in a position corresponding to position 12 in SEQ ID NO:8 or 9. The presence of the target sequence amplified with the second oligonucleotide primer set can be detected by the use of a probe comprising or consisting of at least 10 contiguous nucleotides present in the nucleotide sequence as set forth in SEQ ID NO:7 so that said G/A polymorphism is detected. Preferably, the target sequence amplified with the second oligonucleotide primer set is detected by the use of a probe comprising or consisting of the nucleotide sequence as set forth in SEQ ID NO:8 or 9.

The amplification reaction as defined in the method may further comprise a third oligonucleotide primer set specific for amplifying C. difficile toxin B gene (tcdB). The third oligonucleotide primer set amplifies at least part of nucleotide region as set forth in SEQ ID NO: 10.

Preferably, the third oligonucleotide primer set comprises an oligonucleotide comprising or consisting of at least 10 contiguous nucleotides present in the nucleotide sequence as set forth in SEQ ID NO: 11 and an oligonucleotide comprising or consisting of at least 10 contiguous nucleotides present in the nucleotide sequence as set forth in SEQ ID NO: 12.

More preferably, the third oligonucleotide primer set comprises an oligonucleotide comprising or consisting of the nucleotide sequence as set forth in SEQ ID NO: 11 and an oligonucleotide comprising or consisting of the nucleotide sequence as set forth in SEQ ID NO: 12.

The presence of the target sequence amplified with the third oligonucleotide primer set is detected by the use of a probe comprising or consisting of at least 10 contiguous nucleotides present in the nucleotide sequence as set forth in SEQ ID NO: 13, preferably, by the use of a primer comprising or consisting of the nucleotide sequence as set forth in SEQ ID NO:13.

The present invention is also directed to oligonucleotide primer sets, i.e. oligonucleotides, comprising primers as defined above for the first, second or third oligonucleotide primer set or a mix thereof. The primer sets may also comprise probes as defined above for use with each of the primer sets. The present invention is also directed to the use of these oligonucleotide primer sets for the detection of the presence of a hypervirulent Clostridium difficile strain in a biological sample, such as a stool sample or a food sample.

The present invention also provides kits for detecting a hypervirulent Clostridium difficile strain in a biological sample, a kit may comprise the oligonucleotide primer set as defined above; and a reagent for performing amplification of a nucleic acid. Preferably, the reagent is selected from the group consisting of: DNA polymerase, dNTPs, and a buffer.

Another embodiment of the invention is a method of detecting the presence of a hypervirulent Clostridium difficile strain in a biological sample using oligonucleotide primers and probes with modified nucleotides. Generally, the use of modified nucleotides renders possible shortening of an oligonucleotide primer or probe without compromising its specificity. The amount of modified nucleotides in one primer or probe is preferably 1, 2, 3, 4 or more. The modified nucleotide can be a LNA nucleotide (Exiqon A/S), minor groove binder (MGB™), SuperBase, or Peptide Nucleic Acid (PNA) or any other nucleotide modification having the same effect on the oligonucleotide. The method comprises essentially same steps as the method described above and in the claims but is performed with at least one modified primer or probe. One example of the primers and probes for such method is:
Primer pair 1 (for the detection of hydR gene): SEQ ID NO: 3 and SEQ ID NO: 4 with a probe having the sequence SEQ ID NO: 7.
Primer pair 2 (for the detection of putative conjugative transposon, pct): SEQ ID NO: 5 and SEQ ID NO: 6 with a probe having the sequence CTG TAG ATT TCG GTA CGA (SEQ ID NO: 14), wherein underlined nucleotides are modified nucleotides such as LNA.
Primer pair 3 (for the detection of tedB gene): SEQ ID NO: 11 and SEQ ID NO: 12 with a probe having the sequence SEQ ID NO: 13.

Accordingly, a person skilled in the art would understand that the length of any of the above primers or probes may be shortened in a similar way by using at least one modified nucleotide.

The publications and other materials used herein to illuminate the background of the invention, and in particular, to provide additional details with respect to its practice, are incorporated herein by reference. The present invention is further described in the following example, which is not intended to limit the scope of the invention.

### EXPERIMENTAL SECTION

### Example 1

In this example, the assay of the disclosed invention was used to detect both toxin-producing and non-toxin-producing C. difficile strains. A total of 48 characterized samples representing 37 different ribotypes were tested. This test excluded 027 or genetically very closely related ribotypes.

The assay contains one multiplex PCR reaction which amplifies the target panel (Table 1). Identification of toxin producing C. difficile and differentiation of hypervirulent C. difficile is based on combined detection of these markers. Toxin marker: tcdB gene encodes Toxin B, 027-negative marker: hydR encodes TetR family transcriptional regulator protein and 027-positive marker: pct encodes putative conjugative transposon DNA recombination protein. Primers and probes were as defined in Table 9.

The C. difficile assay should give positive results from different toxin-producing C. difficile strains, and negative results for non-toxin-producing C. difficile strains. Inclusivity (analytical reactivity) is tested to account for potential genetic variation among the targets included in the panel. This example describes the results of the inclusivity of the C. difficile qPCR assay using well characterized strains.

TABLE 1
C. difficile assay target panel

| | Target gene | |
|---|---|---|
| Marker | region | Description |
| Toxin 8 | tcdB | Detects cytotoxin (Toxin B) |
| | | producing C. difficile |
| Positive | pct | Positive hypervirulent marker is |
| hypervirulent | | detected only from hypervirulent |
| marker | | strains (ribotype 027) |
| Negative | hydR | Negative marker is not detected |
| hypervirulent | | from ribotype 027 strains, but is |
| marker | | positive for other C. difficile strains |

### Materials and Methods

### 1.1 the List of the Bacterial Targets

The C. difficile assay covers pathogens causing gastrointestinal infections. A total of 48 characterized samples representing 37 different ribotypes were tested in this inclusivity study covering non-toxinogenic C. difficile and Toxin B producing C. difficile. The list of strains is described in Table 2. This test excluded 027 or genetically very closely related ribotypes.

Strains were collected from commercial available biobanks (ATCC, DSMZ, and Microbiologics). DNA samples were tested in concentrations less than 100 ng/μl.

TABLE 2
Amplidiag C. difficile GE assays inclusivity test panel

| # | Original code |
|---|---|
| 1 | ATCC 51695 |
| 2 | ATCC 43599 |
| 3 | ATCC 17857 |
| 4 | ATCC BAA-1871 |
| 5 | 0329P (ATCC 9689) |
| 6 | ATCC BAA-1813 |
| 7 | ATCC BAA-1874 |
| 8 | ATCC BAA-1809 |
| 9 | ATCC BAA-1810 |
| 10 | ATCC BAA-1801 |
| 11 | ATCC BAA-1382 |
| 12 | ATCC 43596 |
| 13 | ATCC 43600 |
| 14 | AHS 56050 |
| 15 | ATCC 43598 |
| 16 | 106222 |
| 17 | ATCC BAA-1808 |
| 18 | 106216 |
| 19 | ATCC BAA-1812 |
| 20 | ATCC 43601 |
| 21 | ATCC 43602 |
| 22 | 0527P (ATCC 700057) |
| 23 | 106210 |
| 24 | ATCC BAA-1873 |
| 25 | ATCC BAA-1804 |
| 26 | ATCC BAA-1811 |
| 27 | AHS 55375 |
| 28 | 0833P (ATCC 43593) |
| 29 | RHC 7722 |
| 30 | AHS 26782 |
| 31 | AHS 55985 |
| 32 | ATCC BAA-1875 |
| 33 | 106090 |
| 34 | ATCC 43603 |
| 35 | ATCC 43255 |
| 36 | AHS 56035 |
| 37 | ATCC BAA-2156 |
| 38 | RHC 7727 |
| 39 | AHS 55868 |
| 40 | 106194 |
| 41 | RHC 7758 |
| 42 | ATCC BAA-1807 |
| 43 | ATCC BAA-1872 |
| 44 | ATCC BAA-1806 |
| 45 | ATCC BAA 2155 |
| 46 | ATCC BAA-1814 |
| 47 | 106073 |
| 48 | AHS 56010 |

### 1.2 Reagents and Instruments

qPCR Reagents:
qPCR Mastermix, Mobidiag
Assay mixture consisting of C. difficile qPCR primers and probes

### Devices:

Stratagene MxPro 3000

### PCR Setup

### In Reaction:

| | | |
|---|---|---|
| 10 | μl | 2 x Mastermix |
| 5 | μl | 4 x Primer mix |
| 5 | μl | sample/pos. control DNA mix/DNA extraction control/H2O |
| 20 | μl | TOTAL |

### PCR Program:

| | | | |
|---|---|---|---|
| 95° C. | 10 | min | |
| 95° C. | 15 | s | 45x |
| 60° C. | 60 | s | |

### Results

TABLE 3
Identification of markers toxB, pct and hydR in C. difficile strains.

| | | | | Identification of markers | | | |
|---|---|---|---|---|---|---|---|
| # | Original code | Ribotype | Characterization | toxB | pct | hydR | Result |
| 1 | ATCC 5169S | 001 | A+B+, Binary toxin cdtB− | + | − | + | ToxB+ |
| 2 | ATCC 48599 | 001 | A+B+, Binary toxin cdtB− | + | − | + | ToxB+ |
| 3 | ATCC 17857 | 001 | A+B+, Binary toxin cdtB− | + | − | + | ToxB+ |
| 4 | ATCC BAA-1871 | 001 | A+B+, Binary toxin cdtB− | + | − | + | ToxB+ |
| 5 | 0329P (ATCC 9689) | 001 | A+B+, Binary toxin cdtB− | + | − | + | ToxB+ |
| 6 | ATCC BAA-1813 | 002 | A+B+, Binary toxin cdtB− | + | − | + | ToxB+ |
| 7 | ATCC BAA-1874 | 002 | A+B+, Binary toxin cdtB− | + | − | + | ToxB+ |
| 8 | ATCC BAA-1809 | 009 | A−B−, Binary toxin cdtB− | − | − | + | Negative |
| 9 | ATCC BAA-1810 | 009 | A−B−, Binary toxin cdtB− | − | − | + | Negative |
| 10 | ATCC BAA-1801 | 010 | A−B−, Binary toxin cdtB− | − | − | + | Negative |
| 11 | ATCC BAA-1382 | 012 | A+B+, Binary toxin cdtB− | + | − | + | ToxB+ |
| 12 | ATCC 43595 | 012 | A+B+, Binary toxin cdtB− | + | − | + | ToxB+ |
| 13 | ATCC 43600 | 014 | A+B+, Binary toxin cdtB− | + | − | + | ToxB+ |
| 14 | AHS 56050 | 015 | A+B+, Binary toxin−, tcdC 18bp del | + | + | + | ToxB+ |
| 15 | ATCC 43598 | 017 | A−B+, Binary toxin cdtB− | + | − | + | ToxB+ |
| 16 | 106222 | 019 | A+B+, Binary toxin+, tcdC 18bp del | + | − | + | ToxB+ |
| 17 | ATCC BAA-1808 | 020 | A+B+, Binary toxin cdtB− | + | − | + | ToxB+ |
| 18 | 106216 | 023 | A+B+, Binary toxin+ | + | − | + | ToxB+ |
| 19 | ATCC BAA-1812 | 024 | A+B+, Binary toxin cdtB− | + | − | + | ToxB+ |
| 20 | ATCC 43601 | 031 | A−B−, Binary toxin cdtB− | − | − | + | Negative |
| 21 | ATCC 43602 | 031 | A−B−, Binary toxin cdtB− | − | − | + | Negative |
| 22 | 0527P (ATCC 700057) | 038 | A−B−, Binary toxin cdtB− | − | − | + | Negative |
| 23 | 106210 | 045 | A+B+, Binary toxin+ | + | − | + | ToxB+ |
| 24 | ATCC BAA-1873 | 053 | A+B+, Binary toxin cdtB− | + | − | + | ToxB+ |
| 25 | ATCC BAA-1804 | 053 | A+B+, Binary toxin cdtB− | + | − | + | ToxB+ |
| 26 | ATCC BAA-1811 | 057 | A+B+, Binary toxin cdtB− | + | − | + | ToxB+ |
| 27 | AHS 55375 | 058 | A+B+, Binary toxin+ | + | − | + | ToxB+ |
| 28 | 0833P (ATCC 43593) | 060 | A−B−, Binary toxin cdtB− | − | − | + | Negative |
| 29 | RHC 7722 | 063 | A+B+, Binary toxin+, tcdC 18bp del | + | − | + | ToxB+ |
| 30 | AHS 26782 | unk. 67 | A+B+, Binary toxin+, tcdC 18bp del | + | − | + | ToxB+ |
| 31 | AHS 55985 | 075 | A+B+, Binary toxin+, tcdC 18bp del | + | − | − | ToxB+ |
| 32 | ATCC BAA-1875 | 078 | A+B+, Binary toxin cdtB+ | + | − | + | ToxB+ |
| 33 | 106090 | 080 | A+B+, Binary toxin+, tcdC 18bp del | + | − | − | ToxB+ |
| 34 | ATCC 43603 | 085 | A−B−, Binary toxin cdtB− | − | − | + | Negative |
| 35 | ATCC 43255 | 087 | A+B+, Binary toxin cdtB− | + | − | + | ToxB+ |
| 36 | AHS 56035 | 111 | A+B+, Binary toxin+ | + | − | + | ToxB+ |
| 37 | ATCC BAA-2156 | 118 | A+B+, Binary toxin cdtB− | + | − | + | ToxB+ |
| 38 | RHC 7727 | 122 | A+B+, Binary toxin+, tcdC 18bp del | + | − | + | ToxB+ |
| 39 | AHS 55868 | unk. 122 | A+B+, Binary toxin+ | + | − | + | ToxB+ |
| 40 | 106194 | 126 | A+B+, Binary toxin+ | + | − | + | ToxB+ |
| 41 | RHC 7758 | 131 | A+B+, Binary toxin+, tcdC 18bp del | + | − | + | ToxB+ |
| 42 | ATCC BAA-1807 | 140 | A−B−, Binary toxin cdtB− | − | + | + | Negative |
| 43 | ATCC BAA-1872 | 207 | A+B+, Binary toxin cdtB− | + | − | + | ToxB+ |
| 44 | ATCC BAA-1806 | 220 | A+B+, Binary toxin cdtB− | + | − | + | ToxB+ |
| 45 | ATCC BAA-2155 | 251 | A+B+, Binary toxin cdtB+ | + | − | + | ToxB+ |
| 36 | ATCC BAA-1814 | 251 | A+B+, Binary toxin cdtB+ | + | − | + | ToxB+ |
| 47 | 106073 | 254 | A+B+, Binary toxin+ | + | − | + | ToxB+ |
| 48 | AHS 56010 | 308 | A+B+, Binary toxin−, tcdC 18bp del | + | − | + | ToxB+ |

### Functionality of Controls

Positive controls were detected as positive
Negative control was detected as negative
Internal Amplification Control was detected in all samples

### Conclusions

All 39 toxin-producing strains were identified correctly as ToxB+. All 9 non-toxin-producing strains were correctly identified as negative. No strain gave false positive identification of the 027 ribotype (toxB+, pct+, hydR−).

Controls were detected as expected, which confirmed the reliability of the results.

### Example 2

In this example, the functionality of the disclosed invention to differentiate 027 ribotype detection was tested. Two very closely related ribotypes, namely 016 and 176, were included in the samples.

### Materials and Methods

### DNA Extraction

The DNA from C. difficile isolates were extracted as described below:

A colony from bacterial cultures was suspended to the 1×PBS buffer in the final concentration ca. 1.5×10\hat{ }8 CFU/ml (ref. McFarlan standard 0.5). 100 μl of bacterial suspension was transferred to the off-board lysis step following the automated extraction with NucliSENS EasyMAG (bioMérieux) device according to the manufacturer's protocol for Generic 2.0.1 program. DNAs were eluted to the 100 μl of elution buffer. Extraction series contained Extraction Control i.e. C. difficile (non-toxin producing strain).

### Real-Time PCR and Analysis

The PCR reactions were conducted as defined in Example 1. Internal amplification control, Positive PCR control and Negative PCR control is included to the test series.

A total of 18 different 027 ribotype strains, one 016 ribotype strain and one 176 ribotype strain were tested.

TABLE 4
Identification of markers toxB, pct and hydR in C. difficile 027 strains.

| # | Original code | Ribotype | Characterization | toxB | pct | hydR | Result |
|---|---|---|---|---|---|---|---|
| 1 | ATCC BAA-1805 | 027 | A+B+, Binary toxin cdtB+ | + | + | − | 027+ |
| 2 | ATCC BAA-1803 | 027 | A+B+, Binary toxin cdtB+ | + | + | − | 027+ |
| 3 | 01048P (ATCC | 027 | A+B+, Binary toxin cdtB+ | + | + | − | 027+ |
| | BAA-1870) | | | | | | |
| 4 | CD14-038 | 027 | n/a | + | + | − | 027+ |
| 5 | CD13-177 | 027 | n/a | + | + | − | 027+ |
| 6 | CD13-032 | 027 | n/a | + | + | − | 027+ |
| 7 | CD13-221 | 027 | n/a | + | + | − | 027+ |
| 8 | CD14-078 | 027 | n/a | + | + | − | 027+ |
| 9 | CD14-072 | 027 | n/a | + | + | − | 027+ |
| 10 | CD14-161 | 027 | n/a | + | + | − | 027+ |
| 11 | CD13-097 | 027 | n/a | + | + | − | 027+ |
| 12 | CD12-100 | 027 | n/a | + | + | − | 027+ |
| 13 | CD13-305 | 027 | n/a | + | + | − | 027+ |
| 14 | CD13-056 | 027 | n/a | + | + | − | 027+ |
| 15 | CD13-004 | 027 | n/a | + | + | − | 027+ |
| 16 | CD13-247 | 027 | n/a | + | + | − | 027+ |
| 17 | CD13-245 | 027 | n/a | + | + | − | 027+ |
| 18 | CD13-108 | 027 | n/a | + | + | − | 027+ |
| 19 | AHS 55742 | 016 | A+B+, Binary toxin+, tcdC 18bp del | + | + | − | 027+ |
| 20 | AHS 26967 | 176 | A+B+, Binary toxin+, tcdC 18bp del | + | + | − | 027+ |

The assay gave a correct positive identification identification of all the 18 different 027 strains, and gave a positive identification of 016 and 176 ribotypes. Thus, the assay detects genetically closely related 016 and 176 ribotypes in addition to 027 ribotype as 027+.

### Example 3

In this example, the disclosed invention was compared to a prior art method for detecting a 027 presumptive positive C. difficile. The assay of the invention was compared to Xpert C. difficile/Epi (Cepheid) test.

The Xpert C. difficile/Epi test uses the detection of a deletion in tcdC gene to report a positive 027 presumptive finding.

A total of 11 different strains, representing 11 different ribotypes, were tested with both methods and the results were compared.

TABLE 5
Comparison to Xpert C. difficile/Epi (Cepheid) test.

| | | | | | | | | Identification of | | | |
|---|---|---|---|---|---|---|---|---|---|---|---|
| | Original | | | GeneXpert | | | | disclosed markers | | | |
| # | code | Ribotype | Characterization | tox B | Binary | TcdC | Result | toxB | pct | hydR | Result |
| 1 | AHS 55742 | 016 | A+B+, Binary toxin+, tcdC 18bp del | + | + | + | toxigenic C.diff positive, 027 | + | + | − | 027+ |
| | | | | | | | presumptive positive | | | | |
| 2 | 106222 | 019 | A+B+, Binary toxin+, tcdC 18bp del | + | + | + | toxigenic C.diff positive, 027 | + | − | + | ToxB+ |
| | | | | | | | presumptive positive | | | | |
| 3 | AHS 26782 | unk. 67 | A+B+, Binary toxin+, tcdC 18bp del | + | + | + | toxigenic C.diff positive, 027 | + | − | + | ToxB+ |
| | | | | | | | presumptive positive | | | | |
| 4 | 106090 | 080 | A+B+, Binary toxin+, tcdC 18bp del | + | + | + | toxigenic C.diff positive, 027 | + | − | − | ToxB+ |
| | | | | | | | presumptive positive | | | | |
| 5 | AHS 26967 | 176 | A+B+, Binary toxin+, tcdC 18bp del | + | + | + | toxigenic C.diff positive, 027 | + | + | − | 027+ |
| | | | | | | | presumptive positive | | | | |
| 6 | 106210 | 045 | A+B+, Binary toxin+ | + | + | − | toxigenic C.diff positive, 027 | + | − | + | ToxB+ |
| | | | | | | | presumptive negative | | | | |
| 7 | RHC 7722 | 063 | A+B+, Binary toxin+, tcdC 18bp del | + | + | − | toxigenic C.diff positive, 027 | + | − | + | ToxB+ |
| | | | | | | | presumptive negative | | | | |
| 8 | AHS 55985 | 075 | A+B+, Binary toxin+, tcdC 18bp del | + | + | − | toxigenic C.diff positive, 027 | + | − | − | ToxB+ |
| | | | | | | | presumptive negative | | | | |
| 9 | AHS 56035 | 111 | A+B+, Binary toxin+ | + | + | − | toxigenic C.diff positive, 027 | + | − | + | ToxB+ |
| | | | | | | | presumptive negative | | | | |
| 10 | RHC 7727 | 122 | A+B+, Binary toxin+, tcdC 18bp del | + | + | − | toxigenic C.diff positive, 027 | + | − | + | ToxB+ |
| | | | | | | | presumptive negative | | | | |
| 11 | RHC 7758 | 131 | A+B+, Binary toxin+, tcdC 18bp del | + | + | − | toxigenic C.diff positive, 027 | + | − | + | ToxB+ |
| | | | | | | | presumptive negative | | | | |

The Xpert C. difficile/Epi test reported 5 strains to be toxigenic C. difficile positive, 027 presumptive positive, while none of the tested strains were actually ribotype 027. Of these 5 strains, the method of the present invention identified only 2 strains as 027 positive, so demonstrating an improved effect in differentiating between a 027 and non-027 ribotype compared to prior art. It is notable that these two C. difficile strains (016 and 176) have been shown to be highly related to hypervirulent C. difficile strains (Knetsch et al., 2011).

The identification of the disclosed markers reported 9 strains correctly as ToxB+, but not 027+, as expected. In summary, the assay of the invention identified 9/11 strains correctly as 027−, while the Xpert C. difficile/Epi test reported 6/11 strains correctly with regard to the presumptive negativity of 027.

### Example 4

The workflow of the present invention consists of extraction of nucleic acids from stool samples (NucliSens easyMAG), real-time PCR amplification and detection of target gene regions and analysis of results.

In this example, different toxin-producing C. difficile strains were tested as spiked samples in stool background. A total of 35 different strains were used. Each strain was spiked into a stool sample negative for C. difficile. DNA was extracted from stool samples, and qPCR reactions were prepared so that the strain was present in concentrations of either 7.5 CFU/reaction or 75 CFU/reactions as illustrated in Table 6. All samples were tested in duplicate reactions.

The results demonstrate that that the strains were correctly identified as positive in all cases.

TABLE 6
Detection of different toxin-producing C. difficile strains in spiked stool samples.

| | | Cq values of detection of markers | | | | |
|---|---|---|---|---|---|---|
| Original Code | CFU/rxn | toxB | 027+ | 027− | IC | Result |
| ATCC BAA-1870 | 7.5 | 37.14 | 3613 | n/a | 28.32 | 027+ |
| ATCC 9689 | 7.5 | 34.44 | n/a | 36.47 | 27.94 | ToxB+ |
| ATCC BAA-1382 | 7.5 | 36.37 | n/a | 37.44 | 27.77 | ToxB+ |
| ATCC 17858 | 7.5 | 35.32 | n/a | n/a | 28.55 | ToxB4 |
| ATCC 43600 | 7.5 | 37.74 | n/a | 37.21 | 28.68 | ToxB+ |
| ATCC 43596 | 7.5 | 37 | n/a | n/a | 28.29 | ToxB+ |
| ATCC 43594 | 7.5 | 37.89 | n/a | n/a | 28.31 | ToxB+ |
| ATCC 43598 | 7.5 | 36.23 | n/a | n/a | 28.7 | ToxB+ |
| ATCC BAA-1803 | 7.5 | 37.14 | 34.99 | n/a | 28.46 | 027+ |
| ATCC BAA-1808 | 7.5 | 35.7 | n/a | 34.32 | 28.37 | ToxB+ |
| ATCC BAA-1811 | 7.5 | 35.66 | n/a | 35.92 | 28.5 | ToxB+ |
| ATCC BAA-1812 | 7.5 | 37.13 | n/a | 37.67 | 28.43 | ToxB+ |
| ATCC BAA-1813 | 7.5 | 38.07 | n/a | 37.5 | 28.43 | ToxB+ |
| ATCC BAA-1815 | 7.5 | 37.6 | n/a | 36.11 | 28.28 | ToxB+ |
| ATCC BAA-1872 | 7.5 | 35.38 | n/a | 36.6 | 28.36 | ToxB+ |
| ATCC BAA-1875 | 7.5 | 36.84 | n/a | 37.56 | 28.36 | ToxB+ |
| ATCC BAA-2155 | 7.5 | 35.85 | n/a | 35.74 | 28.59 | ToxB+ |
| ATCC BAA-2156 | 7.5 | 35.73 | n/a | 35.67 | 28.3 | ToxB+ |
| ATCC BAA-1804 | 7.5 | 37.37 | n/a | 35.5 | 28.46 | ToxB+ |
| ATCC BAA-1806 | 75 | 35.7 | n/a | 35.33 | 28.97 | ToxB+ |
| CD14-038 | 75 | 36.09 | 35.08 | n/a | 29.12 | 027+ |
| CD13-177 | 75 | 34.53 | 34.21 | n/a | 29.05 | 027+ |
| CD13-032 | 75 | 34.72 | 34.6 | n/a | 29.13 | 027+ |
| CD13-221 | 75 | 36.09 | 37.14 | n/a | 29.42 | 027+ |
| CD14-078 | 75 | 32.07 | 33.3 | n/a | 29.12 | 027+ |
| CD14-072 | 75 | 32.9 | 27.53 | 43.92 | 27.52 | 027+ |
| CD14-161 | 75 | 33.53 | 35.89 | n/a | 29.24 | 027+ |
| CD13-097 | 75 | 32.72 | 33.83 | n/a | 29.14 | 027+ |
| CD12-100 | 75 | 38.56 | 36.27 | n/a | 29.34 | 027+ |
| CD13-305 | 75 | 35.97 | 39.19 | n/a | 29.43 | 027+ |
| CD13-056 | 75 | 35.46 | 35.02 | n/a | 29.14 | 027+ |
| CD13-004 | 75 | 33.22 | 34.07 | n/a | 28.93 | 027+ |
| CD13-247 | 75 | 33.75 | 34.59 | n/a | 29.08 | 027+ |
| CD13-245 | 75 | 33.08 | 34.24 | n/a | 29.02 | 027+ |
| CD13-108 | 75 | 34.55 | 36.18 | n/a | 29.04 | 027+ |

IC = internal control, controls PCR inhibition
CFU/rxn = colony forming units/reaction
Two replicates per sample

### Example 5

This example describes results from a study of potential false positive results in the C. difficile qPCR assay due to a cross-reaction. Sample material for this designed assay is stool sample. Therefore, pathogens (bacteria, viruses and parasites) associated with gastrointestinal infections, and which are not covered by assay panel, can cause potential cross-reaction. Also bacteria included to commensal flora may cross-react. Furthermore, pathogens including to the assay target panel are added to the cross-reaction study since only the target pathogen should be detected and no cross-reaction among other targets should happen.

### Materials and Methods

Reagents, devices and samples
   qPCR Reagents:
Mobidiag's qPCR Mastermix (MM)
Assay mixture consisting of C. difficile qPCR primers and probes (see Table 9)

### Devices:

Stratagene Mxp3000

### PCR Setup

### In Reaction:

| | | |
|---|---|---|
| 10 | μl | 2 x MM |
| 5 | μl | 4 x Primer mix |
| 5 | μl | sample/pos. Control DNA mix/H2O |
| 20 | μl | |

Pos. Control = template mix

| | | | |
|---|---|---|---|
| 95° C. | 10 | min | |
| 95° C. | 15 | s | 40x |
| 60° C. | 1 | min | |

### Samples:

DNA (or RNA) extracted from 127 pathogens. Strains have been mainly collected from commercial available biobanks (ATCC, DSMZ, Microbiologics Qnostics and Vircell). Some strains are added from Mobidiag biobank and those strains have been originally purified from patient samples and characterized by HUSLAB (Helsinki University central hospital laboratory).

The amount of DNA was determined by 16S rRNA assay or by NanoDrop.

TABLE 7
Cross-reaction results.

| # | Species | Result | # | Species, cont. | Result |
|---|---|---|---|---|---|
| 1 | Acinetobacter baumannii | Negative | 65 | Haemophilus parainfluenzae | Negative |
| 2 | Actinomyces actinomycetemcomitans | Negative | 66 | Helicobacter mustelae | Negative |
| 3 | Actinomyces israelii | Negative | 67 | Helicobacter pylori | Negative |
| 4 | Actinomyces naeslundii | Negative | 68 | Helicobacter pylori | Negative |
| 5 | Aspergillus fumigatus | Negative | 69 | Human adenovirus 40 | Negative |
| 6 | Astrovirus | Negative | 70 | Human adenovirus 41 | Negative |
| 7 | Bacillus cereus | Negative | 71 | Human herpesvirus 2 | Negative |
| 8 | Bacillus subtilis | Negative | 72 | Kingella kingae | Negative |
| 9 | Bacteroides fragilis | Negative | 73 | Klebsiella oxytoca | Negative |
| 10 | Bacteroides thetaiotaomicron | Negative | 74 | Klebsiella pneumoniae subsp. pneumoniae | Negative |
| 11 | Bacteroides vulgatus | Negative | 75 | Kluyvera intermedia | Negative |
| 12 | Campylobacter coli | Negative | 76 | Lactobacillus acidophilus | Negative |
| 13 | Campylobacter fetus | Negative | 77 | Lactobacillus casei | Negative |
| 14 | Campylobacter jejuni subsp. jejuni | Negative | 78 | Lactococcus sp. | Negative |
| 15 | Campylobacter lari | Negative | 79 | Listeria monocytogenes | Negative |
| 16 | Candida albicans | Negative | 80 | Micrococcus luteus | Negative |
| 17 | Candida glabrata | Negative | 81 | Moraxella catarrhalis | Negative |
| 18 | Candida krusei | Negative | 82 | Morganella morganii subsp. morganii | Negative |
| 19 | Chromobacterium violaceum | Negative | 83 | Neisseria lactamica | Negative |
| 20 | Citrobacter amalonaticus | Negative | 84 | Neisseria sicca | Negative |
| 21 | Citrobacter braakii | Negative | 85 | Norovirus genogroup 1 | Negative |
| 22 | Citrobacter freundii | Negative | 86 | Norovirus genogroup 2 | Negative |
| 23 | Citrobacter koserii | Negative | 87 | Pasteurella multocida | Negative |
| 24 | Clostridium histolyticum | Negative | 88 | Peptostreptococcus micros | Negative |
| 25 | Clostridium perfringens | Negative | 89 | Plesiomonas shigelloides | Negative |
| 26 | Clostridium septicum | Negative | 90 | Porphyromonas gingivalis | Negative |
| 27 | Clostridium sordellii | Negative | 91 | Prevotella intermedia | Negative |
| 28 | Clostridium sporogenes | Negative | 92 | Prevotella loescheii | Negative |
| 29 | Clostridium tetani | Negative | 93 | Propionibacterium acnes | Negative |
| 30 | Corynebacterium amycolatum | Negative | 94 | Proteus mirabilis | Negative |
| 31 | Corynebacterium diphtheriae | Negative | 95 | Proteus vulgaris | Negative |
| 32 | Cronobacter sakazakii | Negative | 96 | Providencia rettqeri | Negative |
| 33 | Cryptosporidiumn parvum | Negative | 97 | Providencia stuartii | Negative |
| 34 | Cytomegalovirus | Negative | 98 | Pseudomonas aeruginosa | Negative |
| 35 | Desulfovibrio sp. | Negative | 99 | Raoutella ornithinolytica | Negative |
| 36 | Dientamoeba fragilis | Negative | 100 | Rhodococcus equi | Negative |
| 37 | Edwardsiella tarda | Negative | 101 | Rotavirus A | Negative |
| 38 | Eggerthella lenta | Negative | 102 | Saccharomyces kudriaczevii | Negative |
| 39 | Elizabethkingia meningoseptica | Negative | 103 | Salmonella bongori | Negative |
| 40 | Entamoeba histolytica | Negative | 104 | Salmonella enterica subsp. enterica, Typhimurium | Negative |
| 41 | Enterobacter aerogenes | Negative | 105 | Sapovirus | Negative |
| 42 | Enterobacter cloacae | Negative | 106 | Serratia liquefaciens | Negative |
| 43 | Enterobacter hormaechei subsp. hormaechei | Negative | 107 | Serratia marcescens subsp. marcescens | Negative |
| 44 | Enterococcus casseliflavus | Negative | 108 | Shigella boydii | Negative |
| 45 | Enterococcus faecalis | Negative | 109 | Staphylococcus aureus | Negative |
| 46 | Enterococcus faecium | Negative | 110 | Staphylococcus epidermidis | Negative |
| 47 | Enterococcus gallinarum | Negative | 111 | Staphylococcus lugdunensis | Negative |
| 48 | Escherichia coli, non toxigenic | Negative | 112 | Stenotrophomonas maltophilia | Negative |
| 49 | Escherichia coli, EAEC | Negative | 113 | Streptococcus agalactiae | Negative |
| 50 | Escherichia coli, EHEC | Negative | 114 | Streptococcus anginosus | Negative |
| 51 | Escherichia coli, EIEC | Negative | 115 | Streptococcus bovis | Negative |
| 52 | Escherichia coli, EPEC | Negative | 116 | Streptococcus dysgalactiae subsp. equisimilis | Negative |
| 53 | Escherichia coli, ETEC | Negative | 117 | Streptococcus oralis | Negative |
| 54 | Escherichia fergusonii | Negative | 118 | Streptococcus pneumoniae | Negative |
| 55 | Escherichia hermanii | Negative | 119 | Streptococcus pyogenes | Negative |
| 56 | Escherichia vulneris | Negative | 120 | Streptococcus salivarius | Negative |
| 57 | Fusarium solani | Negative | 121 | Streptococcus viridans | Negative |
| 58 | Fusobacterium necrophorum subsp. necrophorum | Negative | 122 | Streptococcus viridans | Negative |
| 59 | Fusobacterium nucleatum subsp. nucleatum | Negative | 123 | Streptomyces spp. | Negative |
| 60 | Gardnerella vaginalis | Negative | 124 | Vibrio parhaemolyticus | Negative |
| 61 | Giardia lamblia | Negative | 125 | Vibrio vulnificus | Negative |
| 62 | Gordonia ssp. | Negative | 126 | Yersinia enterocolitica subsp. enterocolitica | Negative |
| 63 | Haemophilus ducreyi | Negative | 127 | Yersinia pseudotuberculosis | Negative |
| 64 | Haemophilus influenzae | Negative | | | |

Functionality of controls
Positive controls were detected as positive
Negative controls were detected as negative

### Results

The cross-reactivity test showed no false positives.

TABLE 9
Oligonucleotide primers and probes.

| | | Internal | | | |
|---|---|---|---|---|---|
| | | mod- | 5′ mod- | 3′ mod- | |
| Oligo | Sequence | ifica- | ifica- | ifica- | |
| Name | 5′ → 3′ | tion | tion | tion | |
| F_tcdb_ | GGAAGTGAA | | | | SEQ ID |
| 01 | TGTATATGA | | | | NO: 11 |
| | AAACC | | | | |
| R_tcdb_ | GCCATTTTT | | | | SEQ ID |
| 01 | TCTAACTGT | | | | NO: 12 |
| | TTTC | | | | |
| P_tcdb_ | AGAAAGGAG | ZEN | 6-FAM | Iowa | SEQ ID |
| 01_dq | GATATATAA | | | Black ® | NO: 13 |
| | AAGAGTTTT | | | FQ | |
| | AGC | | | | |
| F_hyd_ | CGAACTTCC | | | | SEQ ID |
| 01 | TCTATTAAA | | | | NO: 3 |
| | GC | | | | |
| R_hyd_ | GTGCAATGT | | | | SEQ ID |
| 01 | ATCATCACT | | | | NO: 4 |
| | TTA | | | | |
| P_hyd_ | AATCATTCG | | ROX | Iowa | SEQ ID |
| 01 | CACTATGAA | | | Black ® | NO: 7 |
| | CAACCAATT | | | RQ | |
| F_pct_ | ACGGAAACA | | | | SEQ ID |
| 01 | TCAAATAAC | | | | NO: 5 |
| | G | | | | |
| R_pct_ | GTACCTTTA | | | | SEQ ID |
| 01 | CCAATGTTA | | | | NO: 6 |
| | TTATATG | | | | |
| P_pct_ | TCTGTAGAT | ZEN | HEX | Iowa | SEQ ID |
| 03_dq | TTCGGTACG | | | Black ® | NO: 8 |
| | AAAACTTCA | | | FQ | |
| S_pct_ | TCTGTAGAT | | | Iowa | SEQ ID |
| 03 | TTTGGTACG | | | Black ® | NO: 9 |
| | AAAACTTCA | | | FQ | |

TABLE 10
Amplicons amplified by the oligonucleotide sets.

| | | Size | |
|---|---|---|---|
| Name | Sequence 5′ → 3′ | bp | |
| C.dif_ | ACGGAAACATCAAATAACGAATTGA | 119 | SEQ ID |
| pct_hypV | CAATTTCTGTAGATTTCGGTACGAA | | NO: 2 |
| | AACTTCATGGGAAAGCAGCTTGGTA | | |
| | ACCCAATTAAATGAAATACCATATA | | |
| | ATAACATTGGTAAAGGTAC | | |
| C.dif_ | CGAACTTCCTCTATTAAAGCGAATG | 232 | SEQ ID |
| hydR_01 | GGATTTTTTCTAACCAGCTACAATG | | NO: 1 |
| | TACCATTTTTCTACGTGTGTAATCA | | |
| | TTCGCACTATGAACAACCAATTCTA | | |
| | TTATTTTTTCATTTGCTGTAAGGGT | | |
| | GTCATCAGCAACAAGATACTCTAAA | | |
| | AAATTATTCATTGTGAGTAAAGTTC | | |
| | TTTTGTGACACTTCTCAGTATATCT | | |
| | TCTTTAGTTTTAAAGTGATGATACA | | |
| | TTGCAC | | |
| C.dif_ | GGAAGTGAATGTATATGAAAACCTA | 171 | SEQ ID |
| tcdB_ | AGTAGATATTAGTATATTTTATAAA | | NO: 10 |
| short | TAGAAAGGAGGATATATAAAAGAGT | | |
| | TTTAGCATTTAGATGTAAAAATATT | | |
| | CAATAAAAATATTATAGTAAAGGAG | | |
| | AAAATTTTATGAGTTTAGTTAATAG | | |
| | AAAACAGTTAGAAAAAATGGC | | |

### REFERENCES

Denéve, C., Janoira, C., Poilaneb, I., Fantinatob, C., and Collignon, A., New trends in Clostridium difficile virulence and pathogenesis, International Journal of Antimicrobial Agents, 2009 33:24-28.
Eastwood, K., Else P., Charlett, A., and Wilcox, M H., Comparison of Nine Commercially Available Clostridium difficile Toxin Detection Assays, a Real-Time PCR Assay for C. difficile tedB, and a Glutamate Dehydrogenase Detection Assay to Cytotoxin Testing and Cytotoxigenic Culture Methods, J. Clin. Microbiol., October 2009, p. 3211-3217.
Hirvonen, J J., Mentula, S., Kaukoranta, S-S., Evaluation of a New Automated Homogeneous PCR Assay, GenomEra C. difficile, for Rapid Detection of Toxigenic Clostridium difficile in Fecal Specimens, J. Clin. Microbiol. 2013, 51(9):2908. DOI: 10.1128/JCM.01083-13.
Houser, B A., Hattel, A L., and Jayarao, B M., Real-Time Multiplex Polymerase Chain Reaction Assay for Rapid Detection of Clostridium difficile Toxin-Encoding Strains, Foodborne Pathogens And Disease, 2010, 7(6):719-726.
Knetsch, C W., Hensgens, M P M., Harmanus, C., van der Bijl, M W., Savelkoul, P H M., Kuijper, E J., Corver J., and van Leeuwen, H C., Genetic markers for Clostridium difficile lineages linked to hypervirulence, Microbiology (2011), 157, 3113-3123.
Rupnik, M., Wilcox, M H. and Gerding, D N, Clostridium difficile infection: new developments in epidemiology and pathogenesis, Nature Reviews Microbiology 7, 526-536 (July 2009) p 526, doi: 10.1038/nrmicro2164.

The embodiments of the invention in which an exclusive property or privilege is claimed are defined as follows:

1. An oligonucleotide primer set comprising a first oligonucleotide comprising or consisting of at least 10 contiguous nucleotides present in the nucleotide sequence as set forth in SEQ ID NO:5 and a second oligonucleotide comprising or consisting of at least 10 contiguous nucleotides present in the nucleotide sequence as set forth in SEQ ID NO:6, wherein the primer set amplifies a target sequence in the C. difficile genome, and wherein the primer set further comprises a probe for detecting the amplified target sequence, wherein the probe comprises one or more modified nucleotides and/or a fluorescent label, radiolabel, or phosphorescent label.

2. The oligonucleotide primer set according to claim 1, wherein the first oligonucleotide comprises the nucleotide sequence as set forth in SEQ ID NO:5 and the second oligonucleotide comprises the nucleotide sequence as set forth in SEQ ID NO:6.

3. The oligonucleotide primer set according to claim 2, wherein the first oligonucleotide consists of the nucleotide sequence as set forth in SEQ ID NO:5 and the second oligonucleotide consists of the nucleotide sequence as set forth in SEQ ID NO:6.

4. The oligonucleotide primer set according to claim 1, wherein the probe comprises at least 10 contiguous nucleotides present in the nucleotide sequence as set forth in SEQ ID NO:8 or SEQ ID NO:9.

5. The oligonucleotide primer set according to claim 4, wherein the probe comprises the nucleotide sequence as set forth in SEQ ID NO:8 or SEQ ID NO:9.

6. The oligonucleotide primer set according to claim 1, further comprising a second oligonucleotide primer set, wherein the second oligonucleotide primer set comprises a third oligonucleotide comprising or consisting of at least 10 contiguous nucleotides present in the nucleotide sequence as set forth in SEQ ID NO:3 and a fourth oligonucleotide comprising or consisting of at least 10 contiguous nucleotides present in the nucleotide sequence as set forth in SEQ ID NO:4, wherein the second oligonucleotide primer set amplifies a target sequence in the C. difficile hydR gene.

7. The oligonucleotide primer set according to claim 6, wherein the third oligonucleotide comprises or consists of the nucleotide sequence as set forth in SEQ ID NO:3 and the fourth oligonucleotide comprises or consists of the nucleotide sequence as set forth in SEQ ID NO:4.

8. The oligonucleotide primer set according to claim 6, further comprising a probe for detecting the amplified hydR target sequence, said probe comprising at least 10 contiguous nucleotides present in the nucleotide sequence as set forth in SEQ ID NO:7.

9. The oligonucleotide primer set according to claim 8, wherein the probe for detecting the amplified hydR target sequence comprises the nucleotide sequence as set forth in SEQ ID NO:7.

10. The oligonucleotide primer set according to claim 6, further comprising a third oligonucleotide primer set, wherein the third oligonucleotide primer set comprises a fifth oligonucleotide comprising or consisting of at least 10 contiguous nucleotides present in the nucleotide sequence as set forth in SEQ ID NO:11 and a sixth oligonucleotide comprising or consisting of at least 10 contiguous nucleotides present in the nucleotide sequence as set forth in SEQ ID NO:12, wherein the third oligonucleotide primer set amplifies a target sequence in the C. difficile tcdB gene.

11. The oligonucleotide primer set according to claim 10, wherein the fifth oligonucleotide comprises or consists of the nucleotide sequence as set forth in SEQ ID NO:11 and the sixth oligonucleotide comprises or consists of the nucleotide sequence as set forth in SEQ ID NO:12.

12. The oligonucleotide primer set according to claim 10, further comprising a probe for detecting the amplified tcdB target sequence, said probe comprising at least 10 contiguous nucleotides present in the nucleotide sequence as set forth in SEQ ID NO:13.

13. The oligonucleotide primer set according to claim 12, wherein the probe for detecting the amplified tedB target sequence comprises the nucleotide sequence as set forth in SEQ ID NO:13.

14. A kit for detecting a hypervirulent C. difficile strain in a biological sample, the kit comprising:
(i) an oligonucleotide primer set comprising a first oligonucleotide comprising or consisting of at least 10 contiguous nucleotides present in the nucleotide sequence as set forth in SEQ ID NO:5 and a second oligonucleotide comprising or consisting of at least 10 contiguous nucleotides present in the nucleotide sequence as set forth in SEQ ID NO:6, wherein the primer set amplifies a target sequence in the C. difficile genome;
(ii) a probe for detecting the amplified target sequence, wherein the probe comprises one or more modified nucleotides and/or a fluorescent label, radiolabel, or phosphorescent label; and
(iii) a reagent for performing amplification of a nucleic acid.

15. The kit of claim 14, wherein the reagent is selected from the group consisting of a DNA polymerase, dNTPs, and a buffer.

16. The kit of claim 14, wherein the first oligonucleotide comprises or consists of the nucleotide sequence as set forth in SEQ ID NO:5 and the second oligonucleotide comprises or consists of the nucleotide sequence as set forth in SEQ ID NO:6.

17. The kit of claim 14, wherein the probe comprises at least 10 contiguous nucleotides present in the nucleotide sequence as set forth in SEQ ID NO:8 or SEQ ID NO:9.

18. The kit of claim 14, further comprising a second oligonucleotide primer set, wherein the second oligonucleotide primer set comprises a third oligonucleotide comprising or consisting of at least 10 contiguous nucleotides present in the nucleotide sequence as set forth in SEQ ID NO:3 and a fourth oligonucleotide comprising or consisting of at least 10 contiguous nucleotides present in the nucleotide sequence as set forth in SEQ ID NO:4, wherein the second oligonucleotide primer set amplifies a target sequence in the C. difficile hydR gene.

19. The kit of claim 18, wherein the third oligonucleotide comprises or consists of the nucleotide sequence as set forth in SEQ ID NO:3 and the fourth oligonucleotide comprises or consists of the nucleotide sequence as set forth in SEQ ID NO:4.

20. The kit of claim 18, further comprising a third oligonucleotide primer set, wherein the third oligonucleotide primer set comprises a fifth oligonucleotide comprising or consisting of at least 10 contiguous nucleotides present in the nucleotide sequence as set forth in SEQ ID NO:11 and a sixth oligonucleotide comprising or consisting of at least 10 contiguous nucleotides present in the nucleotide sequence as set forth in SEQ ID NO:12, wherein the third oligonucleotide primer set amplifies a target sequence in the C. difficile tcdB gene.

21. The kit of claim 20, wherein the fifth oligonucleotide comprises or consists of the nucleotide sequence as set forth in SEQ ID NO:11 and the sixth oligonucleotide comprises or consists of the nucleotide sequence as set forth in SEQ ID NO: 12.


SEQUENCE LISTING (13 sequences)

SEQ ID NO: 1 - DNA, 232 bp, Clostridium difficile
  source 1..232: mol_type = genomic DNA; strain = 630
cgaacttcctctattaaagcgaatgggattttttctaaccagctacaatgtaccatttttctacgtgtgtaatcattcgcactatgaacaaccaattctattattttttcatttgctgtaagggtgtcatcagcaacaagatactctaaaaaattattcatttgtgagtaaagttcttttgtgacacttctcagtatatcttctttagttttaaagtgatgatacattgcac

SEQ ID NO: 2 - DNA, 119 bp, Clostridium difficile
  source 1..119: mol_type = genomic DNA; strain = 630
acggaaacatcaaataacgaattgacaatttctgtagatttcggtacgaaaacttcatgggaaagcagcttggtaacccaattaaatgaaataccatataataacattggtaaaggtac

SEQ ID NO: 3 - DNA, 20 bp, Clostridium difficile
  source 1..20: mol_type = genomic DNA; strain = 630
cgaacttcctctattaaagc

SEQ ID NO: 4 - DNA, 21 bp, Clostridium difficile
  source 1..21: mol_type = genomic DNA; strain = 630
gtgcaatgtatcatcacttta

SEQ ID NO: 5 - DNA, 19 bp, Clostridium difficile
  source 1..19: mol_type = genomic DNA; strain = 630
acggaaacatcaaataacg

SEQ ID NO: 6 - DNA, 25 bp, Clostridium difficile
  source 1..25: mol_type = genomic DNA; strain = 630
gtacctttaccaatgttattatatg

SEQ ID NO: 7 - DNA, 27 bp, Clostridium difficile
  source 1..27: mol_type = genomic DNA; strain = 630
aatcattcgcactatgaacaaccaatt

SEQ ID NO: 8 - DNA, 27 bp, Clostridium difficile
  source 1..27: mol_type = genomic DNA; strain = 630
tctgtagatttcggtacgaaaacttca

SEQ ID NO: 9 - DNA, 27 bp, Clostridium difficile
  source 1..27: mol_type = genomic DNA; strain = 630
tctgtagattttggtacgaaaacttca

SEQ ID NO: 10 - DNA, 171 bp, Clostridium difficile
  source 1..171: mol_type = genomic DNA; strain = 630
ggaagtgaatgtatatgaaaacctaagtagatattagtatattttataaatagaaaggaggatatataaaagagttttagcatttagatgtaaaaatattcaataaaaatattatagtaaaggagaaaattttatgagtttagttaatagaaaacagttagaaaaaatggc

SEQ ID NO: 11 - DNA, 23 bp, Clostridium difficile
  source 1..23: mol_type = genomic DNA; strain = 630
ggaagtgaatgtatatgaaaacc

SEQ ID NO: 12 - DNA, 22 bp, Clostridium difficile
  source 1..22: mol_type = genomic DNA; strain = 630
gccattttttctaactgttttc

SEQ ID NO: 13 - DNA, 30 bp, Clostridium difficile
  source 1..30: mol_type = genomic DNA; strain = 630
agaaaggaggatatataaaagagttttagc